(19) United States
(12) Patent Application Publication
(10) Pub. No.: US 2025/0135211 A1
(43) Pub. Date: May 1, 2025

(54) IMPLANTABLE MEDICAL DEVICE FEEDTHROUGH ASSEMBLY WITH ELECTRICALLY INSULATIVE OXIDE SURFACE COATING

(71) Applicant: Medtronic, Inc., Minneapolis, MN (US)

(72) Inventors: Sadegh Behdad, Apple Valley, MN (US); Joachim Hossick-Schott, Minneapolis, MN (US); Rajesh V. Iyer, Eden Prairie, MN (US); Andrew J. Thom, Maple Grove, MN (US)

(21) Appl. No.: 18/925,265

(22) Filed: Oct. 24, 2024

Related U.S. Application Data
(60) Provisional application No. 63/545,679, filed on Oct. 25, 2023.

(51) Int. Cl. A61N 1/375

(52) U.S. Cl. CPC A61N 1/3754

(57) ABSTRACT

Feedthroughs, feedthrough assemblies, and methods of making the same for use in implantable medical devices are provided herein. Feedthroughs provided herein include a feedthrough ferrule, a feedthrough pin, and an electrically insulative metal oxide surface coating disposed on the feedthrough ferrule, disposed on the feedthrough pin, or disposed on both the feedthrough pin and the feedthrough ferrule.

### RELATED APPLICATIONS

This application claims the benefit of U.S. Provisional Application Ser. No. 63/545,679, filed Oct. 25, 2023, the entire contents of each of which are incorporated herein by reference.

### TECHNICAL FIELD

This disclosure generally relates to, among other things, feedthrough assemblies for use in implantable medical devices and, more specifically, feedthrough assemblies with electrically insulative metal oxide surface coating and method of making the same.

### BACKGROUND

Various systems require electrical coupling between electrical devices disposed within a sealed enclosure or housing and devices or systems external to the enclosure. Oftentimes, such electrical coupling needs to withstand various environmental factors such that a conductive pathway or pathways from the external surface of the enclosure to within the enclosure remains stable. For example, implantable medical devices (IMDs), e.g., cardiac pacemakers, defibrillators, neurostimulators, and drug pumps, which include electronic circuitry and one or more power sources, require an enclosure or housing to contain and seal these elements within a body of a patient. Many of these IMDs include one or more electrical components such as, for example, feedthrough assemblies to provide electrical connections between the elements contained within the housing and components of the IMD external to the housing, for example, one or more sensors, electrodes, and lead wires mounted on an exterior surface of the housing, or electrical contacts housed within a connector header, which is mounted on the housing to provide coupling for one or more implantable leads. Feedthroughs may be described as an apparatus that provides electrical coupling between electrical devices disposed within a sealed enclosure or housing and devices or systems external to the enclosure in an electrically-insulated and hermetically-sealed manner.

Implantable medical devices generally use insulating plugs and potting material compositions to bond feedthrough pins within a feedthrough ferrule and to electrically insulate the pin from the ferrule. Potting materials and insulating plugs may also commonly be used to isolate the internal components of the implantable medical device from the patient's body and the patient's bodily fluid. Because internal components, such as batteries, capacitors, and processors, for example, may commonly be sensitive to the aqueous environment of the patient's body, it is important to isolate sensitive internal components.

In some cases, electrical short pathways may arise between the feedthrough pin and the feedthrough ferrule. To isolate, or hermetically seal, the internal components of an implantable medical device and maintain electrical insulation between the feedthrough pin and the feedthrough ferrule, particularly for implantable medical devices intended for long-term use, there is a need for improved electrical insulation in feedthrough assemblies.

### SUMMARY

As described herein, feedthrough assemblies may be provided with an electrically insulative metal oxide surface coating disposed between and providing electrical insulation between the feedthrough pin and the feedthrough ferrule. Electrically insulative metal oxide surface coatings provided herein may be described as providing electrical insulation between the feedthrough pin and the feedthrough ferrule, such electrical insulation being additional to, supplementary to, or enhancing electrical insulation provided by an electrically insulative plug.

Embodiments disclosed herein may include an implantable medical device feedthrough assembly having a substrate with a first major surface configured to be positioned towards an internal volume of the implantable medical device, an opposing second major surface configured to be positioned away from the internal volume of the implantable medical device, a feedthrough ferrule with a ferrule sidewall defining a ferrule lumen extending through the substrate from the first major surface to the second major surface, a feedthrough pin disposed within the ferrule lumen and configured to electrically connect a component in the internal volume of the implantable medical device with a component in communication with an environment external to the implantable medical device, at least one metal oxide surface coating on a portion of at least one of the ferrule sidewall or a surface of the feedthrough pin to provide electrical insulation between the feedthrough pin and the feedthrough ferrule, a potting material disposed between and providing electrical insulation between the feedthrough pin and the feedthrough ferrule, and an insulating plug disposed at least partially within the feedthrough ferrule and positioned to separate the potting material from the internal volume. The potting material may be positioned to separate the internal volume from the external environment. The metal oxide surface coating may be disposed between the ferrule sidewall and the surface of the feedthrough pin, and may extend along at least one of the ferrule sidewall or the surface of the feedthrough pin from the second major surface of the substrate towards the first major surface of the substrate. The metal oxide surface coating may extend along the ferrule sidewall in the ferrule lumen from a major surface of the insulating plug towards the second major surface of the substrate. The metal oxide surface coating may be disposed on the ferrule sidewall. The metal oxide surface coating may be disposed on the surface of the feedthrough pin. At least one metal oxide surface coating may have a first metal oxide surface coating disposed on the surface of the feedthrough pin, and a second metal oxide surface coating disposed on the ferrule sidewall. The feedthrough pin may be or include titanium, niobium, aluminum, tantalum, zirconium, hafnium, or a combination thereof. The feedthrough ferrule may be or include titanium, niobium, aluminum, tantalum, or a combination thereof. The metal oxide surface coating may be or include titanium oxide, niobium oxide, aluminum oxide, tantalum oxide, zirconium oxide, hafnium oxide or a combination thereof. The metal oxide surface coating may have a thickness of 10 nm or greater, 20 nm or greater, 30 nm or greater, 50 nm or greater, or 100 nm or greater measured in a direction perpendicular to a longitudinal axis of the feedthrough pin. The metal oxide surface coating may have a thickness of between 50 nm and 300 nm or between 100 nm and 200 nm measured in a direction perpendicular to a longitudinal axis of the feedthrough pin. The insulating plug may be or include glass, sapphire, alumina, or a combination thereof.

Embodiments disclosed herein may additionally or alternatively include an implantable medical device having the feedthrough assembly described herein, a housing defining the internal volume of the implantable medical device, an internal component electrically coupled to the feedthrough pin and positioned in the internal volume, and a second component electrically coupled to the feedthrough pin and in communication with the external environment. The feedthrough assembly may be fixed relative to the housing. The implantable medical device may further include a connector header configured to receive a medical lead. The second component may be an electrical contact within the connector header, and the electrical contact may be positioned and configured to electrically couple the lead to the internal component via the feedthrough pin.

Embodiments disclosed herein may additionally or alternatively include a method for forming an implantable medical device feedthrough assembly, the method including fixing a feedthrough pin within a feedthrough ferrule with an insulating plug; oxidizing one or both of the feedthrough pin and the feedthrough ferrule, thereby forming at least one metal oxide surface coating on a portion of at least one of the ferrule sidewall or a surface of the feedthrough pin to provide electrical insulation between the feedthrough pin and the feedthrough ferrule; and bonding the pin within the ferrule with a potting material. The potting material may provide electrical insulation between the feedthrough pin and the feedthrough ferrule. The oxidizing may occur at an ambient temperature of between 200° C. and 500° C. or between 150° C. and 450° C. The oxidizing may include applying a voltage across the feedthrough pin. The voltage may be between 50 V and 400 V or between 100 V and 450 V. The oxidizing may include applying a voltage across the feedthrough ferrule. The voltage may be between 50 V and 400 V or between 100 V and 450 V. The metal oxide surface coating may be or include titanium oxide, niobium oxide, aluminum oxide, tantalum oxide, or a combination thereof. The metal oxide surface coating may have or include a thickness of 10 nm or greater, 20 nm or greater, 30 nm or greater, 50 nm or greater, or 100 nm or greater measured in a direction perpendicular to a longitudinal axis of the feedthrough pin. The metal oxide surface coating may have or include a thickness of between 50 nm and 300 nm or between 100 nm and 200 nm measured in a direction perpendicular to a longitudinal axis of the feedthrough pin. The metal oxide surface coating may be formed on the surface of the feedthrough pin. The metal oxide surface coating may be formed on the ferrule sidewall. The feedthrough pin may be or include titanium, niobium, aluminum, tantalum, or a combination thereof. The feedthrough ferrule may be or include titanium, niobium, aluminum, tantalum, or a combination thereof.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1A is an illustrative implantable medical device utilizing a feedthrough assembly with an electrically insulative metal oxide surface coating implanted in a patient.

FIGS. 1B and 1C are various schematic views of the illustrative implantable medical device of FIG. 1A. FIG. 1B is a schematic perspective view of the implantable medical device and FIG. 1C is a schematic exploded view of the implantable medical device.

FIG. 2 is a cross-section side view of an illustrative feedthrough assembly of the implantable medical device of FIGS. 1A-C, including the metal oxide surface coating disposed on a surface of a feedthrough pin of the feedthrough assembly.

FIG. 3 is a cross-section side view of another illustrative feedthrough assembly of the implantable medical device of FIGS. 1A-C, with the metal oxide surface coating disposed on a surface of a feedthrough ferrule of the feedthrough assembly.

FIG. 4 is a cross-section side view of still another illustrative feedthrough assembly of the implantable medical device of FIGS. 1A-C, with a first metal oxide surface coating disposed on a surface of the feedthrough pin and a second metal oxide surface coating disposed on a surface of a feedthrough ferrule.

FIG. 5 is a schematic of an illustrative method for preparing the feedthrough assembly of FIGS. 2-4.

The figures are rendered primarily for clarity and, as a result, are not necessarily drawn to scale. Moreover, various structure/components may be shown diagrammatically or removed from some of or all the views to better illustrate aspects of the depicted embodiments, or where inclusion of such structure/components is not necessary to an understanding of the various exemplary embodiments described herein. The lack of illustration/description of such structures/components in a particular figure is, however, not to be interpreted as limiting the scope of the various embodiments in any way.

### DETAILED DESCRIPTION

All scientific and technical terms used herein have meanings commonly used in the art unless, otherwise specified. The definitions provided herein are to facilitate understanding of certain terms used frequently herein and are not meant to limit the scope of the present disclosure.

Unless otherwise indicated, the terms “polymer”, “polymerized monomers”, and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

In this disclosure, all numbers are assumed to be modified by the term “about,” which encompasses the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively unless the context specifically refers to a disjunctive use.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to”, “at most”, or “at least” a particular value, that value is included within the range.

As used here, “have,” “having,” “include,” “including,” “comprise,” “comprising,” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method, or the like, means that the components of the composition, product, method, or the like are limited to the enumerated components and any other components that do not materially affect the basic and novel characteristic(s) of the composition, product, method, or the like.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure. Such inclusive or open-ended words encompass more restrictive or closed terms or phrases, such as “consisting” or “consisting essentially.”

Reference in the specification to “some embodiments,” “an embodiment,” “one embodiment,” “embodiments,” “one or more embodiments,” or “other embodiments” means that a particular feature, structure, or characteristic described in connection with the embodiments is included in at least some embodiments, but not necessarily all embodiments, of the present disclosures.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

In several places throughout the application, guidance is provided through examples, which examples, including the particular aspects thereof, can be used in various combinations and be the subject of claims. In each instance, the recited list serves only as a representative group and should not be interpreted as an exclusive list. It is to be understood that the particular examples, materials, amounts, and procedures are to be interpreted broadly in accordance with the scope and spirit of the invention as set forth herein.

Reference will now be made in greater detail to various embodiments of the subject matter of the present disclosure, one or more embodiments of which are illustrated in the accompanying drawings. Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same as or similar to other numbered components.

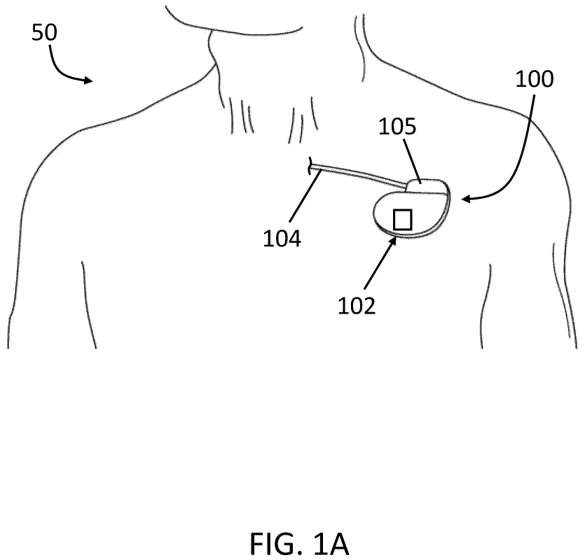

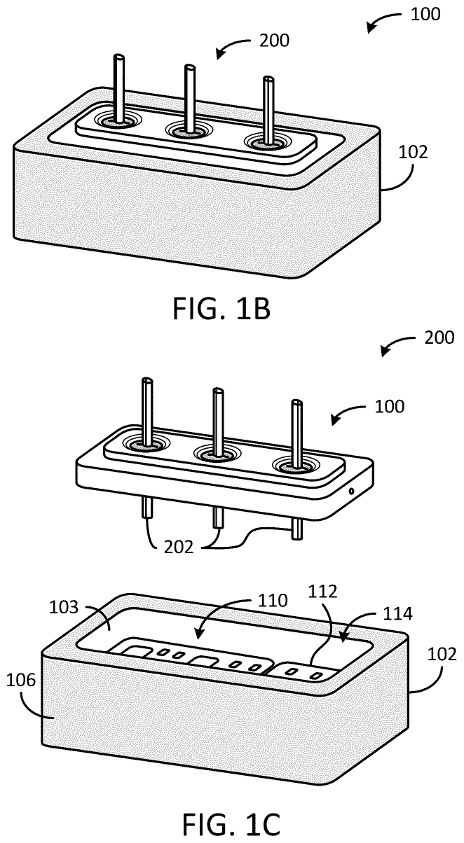

As described herein, a feedthrough assembly with an electrically insulative metal oxide surface coating may be provided, for example, for use in an implantable medical device. An illustrative implantable medical device 100 implanted in a patient 50 including the feedthrough assembly with the electrically insulative metal oxide surface coating is shown in FIG. 1A. The implantable medical device 100 may include a housing 102, which may also be referred to as a case. The housing 102 may define an internal volume of the implantable medical device 100 and may house one or more internal components of the implantable medical device 100, such as one or more batteries, electrical circuits, and the like. The feedthrough assembly may be fixed relative to the housing 102.

The implantable medical device may be configured to be electrically connectable with one or more external components, such as a medial lead 104. The one or more external components may be electrically connected to the one or more internal components via a feedthrough, which may be positioned in a connector header 105. The connector header 105 may form a unitary part with, or may be coupled to, the housing 102. The connector header 105 may be configured to receive the one or more external components, such as the lead 104. For example, the connector header 105 may define a bore through which a portion of the lead 104 may be inserted. The lead 104 may have electrical contacts that electrically couple with respective electrical contacts in the connector header 105 when the lead 104 is inserted into the bore. The electrical contacts in the connector header 105 may be electrically coupled to the electrical components of the implantable medical device 100 through one or more feedthroughs.

Various schematic views of an illustrative embodiment of the implantable medical device 100 (without the connection header 105) that may utilize one or more feedthrough assemblies as described herein are shown in FIGS. 1B and 1C. FIG. 1B is a schematic perspective view of the implantable medical device 100 and FIG. 1C is a schematic exploded view of the implantable medical device 100. The implantable medical device 100 may include the housing 102, which may include an inner surface 104 and an outer surface 106. The implantable medical device 100 may also include one or more internal components disposed in the housing 102, such as at least one electronic device 110 and/or a power source 112. In one or more embodiments, the power source 112 can be disposed within a cavity 114 of the housing 102. The power source 112 may include one or more power source contacts that can be operatively coupled to the electronic device 110 or any of one or more feedthrough pins 202.

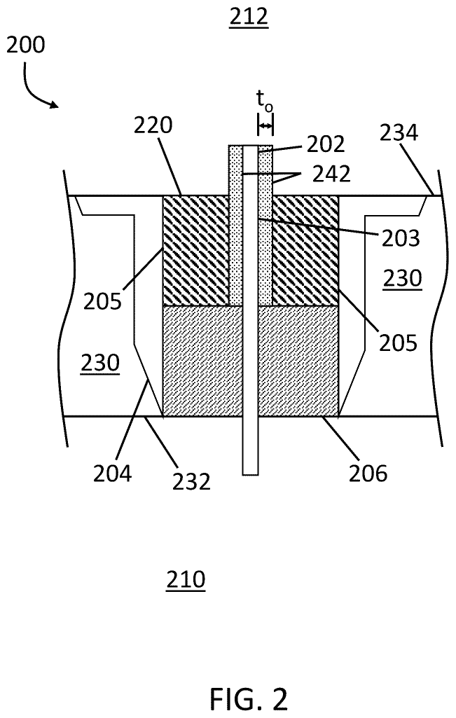

An illustrative feedthrough assembly 200 including at least one metal oxide surface coating 242 that operates as an electrically insulative oxide layer is shown in FIG. 2. In other words, the metal oxide surface coating 242 may be described as having electrically insulative properties. The feedthrough assembly 200 may include a substrate 230 having a first major surface 232 and an opposing second major surface 234. The substrate 230 may form a part of an implantable medical device (e.g., the implantable medical device 100), such as a part of the housing (e.g., the housing 102). The first major surface 232 may be configured to be positioned towards an internal volume 210 (such as towards the cavity 114 within the housing 102 shown in FIG. 1C) of the implantable medical device. The second major surface 234 may be configured to be positioned away from the internal volume 210 of the implantable medical device, which may be toward an external environment 212 (such as within the connector header 105 shown in FIG. 1A, which may define a bore in communication with a patient's bodily fluid).

The feedthrough assembly 200 may include one or more feedthrough ferrules, such as the illustrative feedthrough ferrule 204 shown in FIGS. 2-4. The feedthrough ferrule 204 may define a surface (a ferrule sidewall 205) that defines a ferrule lumen extending through the substrate 230 from the first major surface 232 to the second major surface 234. At least a portion of the feedthrough ferrule 204 may be disposed between the internal volume 210 and the external environment 212. The feedthrough ferrule 204 may be tubular.

The feedthrough assembly 200 may further include a feedthrough pin 202. The feedthrough pin 202 may be disposed within and extend through the ferrule lumen. For example, the feedthrough pin 202 may be coaxially disposed within and extend through the ferrule lumen. The feedthrough pin 202 may be configured to electrically connect a component in the internal volume 210 of the implantable medical device, (such as batteries, capacitors, and/or processors, for example) with a component (such as implantable leads, for example) in communication with an environment external to the implantable medical device.

The implantable medical device may further include at least a second component, such as an electrical contact in the connector header (e.g., the connector header 105 shown in FIG. 1A) in communication with the external environment (e.g., the external environment 212). The electrical contact in the connector header (i.e., the second component) may be electrically coupled to a respective feedthrough pin of the one or more feedthrough pins (e.g., the feedthrough pin 202). For example, the second component may be electrically coupled, or electrically couplable, to a lead (e.g., the medical lead 104) or to an electrical contact of a lead. In other words, a component disposed, or positioned, in the internal volume of the implantable medical device (i.e., an internal component) may be electrically coupled to a respective component disposed, or positioned, outside of the internal volume of the implantable medical device (e.g., in the connector header 105 or in the external environment 212) via a respective feedthrough pin (e.g., the feedthrough pin 202).

The feedthrough assembly 200 may include a potting material 220 disposed between and providing electrical insulation between the feedthrough pin 202 and the feedthrough ferrule 204, e.g., aligned coaxially between the feedthrough pin 202 and sidewall 205 of the ferrule 204. The potting material 220 may be positioned to help separate elements of the feedthrough assembly 200 from the external environment 212. For example, the potting material 220 may be positioned to help separate the internal volume 210, portions of the feedthrough pin 202, and/or portions of the feedthrough ferrule 204 from the external environment 212. More specifically, the potting material 220 may be positioned to help separate elements, or portions of elements, of the feedthrough assembly 200 from the patient's body and the patient's bodily fluid.

The feedthrough assembly 200 may include an insulating plug 206 disposed at least partially within the feedthrough ferrule 204. The insulating plug 206 may be configured and positioned to provide a hermetic seal between the external environment 212 and the internal volume 210. The insulating plug 206 may be positioned to separate the potting material 220 from the internal volume 210. The feedthrough pin 202 may extend through a lumen in the insulating plug 206.

The feedthrough assembly 200 may include at least one metal oxide surface coating 242 disposed between and providing electrical insulation between the feedthrough pin 202 and the feedthrough ferrule 204. Without wishing to be bound by theory, the metal oxide surface coating 242 may provide additional, or secondary, insulation between the feedthrough pin 202 and the feedthrough ferrule 204. More specifically, and still without wishing to be bound by theory, the metal oxide surface coating 242 may provide improved electrical insulation between the feedthrough pin 202 and the feedthrough ferrule 204 along the boundary between the contacting surfaces of the potting material 220 and the insulative plug 206, along the exposed surface of potting material 220, or both. More specifically, the metal oxide surface coating 242 may provide improved electrical insulation between the feedthrough pin 202 and the feedthrough ferrule 204 along the perimeter of the feedthrough pin 202 at the boundary between the potting material 220 and the insulative plug 206 and/or along the perimeter of the feedthrough ferrule 202 at the boundary between the potting material 220 and the insulative plug 206. Without wishing to be bound by theory, electrical insulation provided by the metal oxide surface coating 242, as described herein, may be useful, for example, if an electrical short pathway forms between the feedthrough pin 242 and the feedthrough ferrule 206 along the boundary between the potting material 220 and the insulative plug 206. The metal oxide surface coating 242 may extend along the ferrule sidewall 205 in the ferrule lumen from the second major surface 234 towards the first major surface 232. In some embodiments, the metal oxide surface coating 242 may extend along the ferrule sidewall 205 in the ferrule lumen from the second major surface 234 towards but not all the way to the first major surface 232. Additionally or alternatively, the metal oxide surface coating 242 may extend along ferrule sidewall 205 in the ferrule lumen from the second major surface 234 to the insulating plug 206. In other words, the metal oxide surface coating 242 may extend in the ferrule lumen from the second major surface 234 toward the first major surface 232 and to the insulating plug 206. In one or more embodiments, the metal oxide surface coating 242 does not extend along the contact surfaces between the insulating plug 206 and the ferrule sidewall 205 and/or along the contact surfaces between the insulating plug 206 and the feedthrough pin 202.

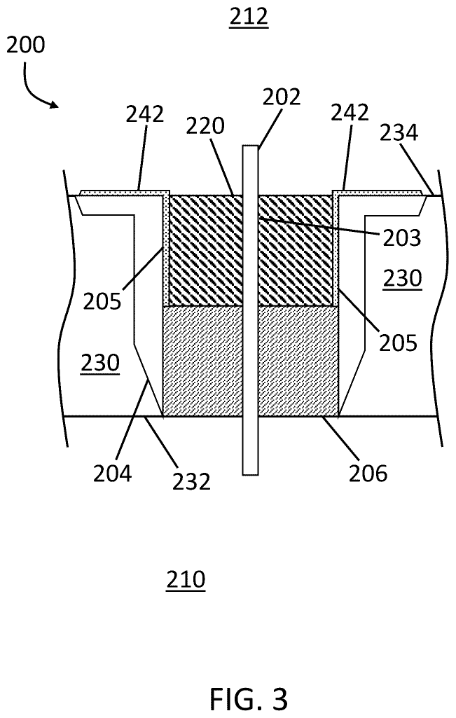

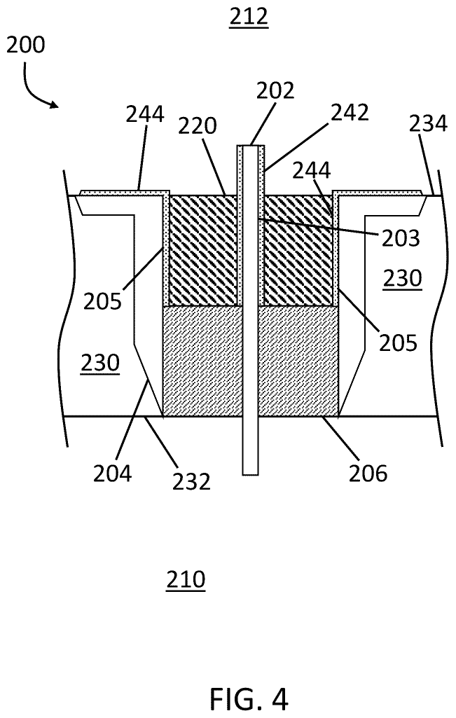

The feedthrough assembly 200 may include at least one metal oxide surface coating (e.g., the metal oxide surface coating 242) forming an electrically insulative oxide layer. As illustrated in FIG. 2, the metal oxide surface coating 242 may be disposed on a surface 203 of the feedthrough pin 202. Additionally or alternatively, the metal oxide surface coating 242 may be disposed on the ferrule sidewall 205 of the feedthrough ferrule 204, as shown in FIG. 3. In some embodiments, and as shown in FIG. 4, at least one metal oxide surface coating may include a first metal oxide surface coating 242 disposed on a surface 203 of the feedthrough pin 202 and a second metal oxide surface coating 244 disposed on the ferrule sidewall 205 of the feedthrough ferrule 204.

In one or more embodiments as described herein, the implantable medical device feedthrough assembly may include a feedthrough pin (e.g., the feedthrough pin 202 of the implantable medical device feedthrough assembly 200 shown in FIG. 2). The feedthrough pin may be or include any suitable materials or combination of materials. Suitable feedthrough pin materials may be selected based on electrical conductivity, biocompatibility, desired metal oxide surface coating, anodic oxidation capabilities (found, for example, in metals from the valve-metals group, including Nb, Ta, Al, Ti, Zr, and Hf), material compatibility with the insulating plug (e.g., the insulating plug 206), or material compatibility with the potting material (e.g., the potting material 220), as examples. Examples of suitable feedthrough pin materials may include titanium, niobium, aluminum, tantalum, zirconium, hafnium, or a combination thereof. Still further examples of suitable feedthrough pin materials may include metal alloys, such as an alloy of aluminum or alloys of suitable feedthrough pin materials described herein. It will be understood in light of the present disclosure that any suitable feedthrough pin material or combination of materials may be used, and the disclosure is not limited in this regard. It will be further understood in light of the present disclosure that suitable feedthrough pin materials may vary depending on factors, including those described herein.

In at least one embodiment as described herein, the implantable medical device feedthrough assembly may include a feedthrough ferrule (e.g., the feedthrough ferrule 204 of the implantable medical device feedthrough assembly 200 shown in FIG. 2). The feedthrough ferrule may be or include any suitable materials or combination of materials. Suitable feedthrough ferrule materials may be selected based on biocompatibility, electrical conductivity, desired metal oxide surface coating, anodic oxidation capabilities (found, for example, in metals from the valve-metals group, including Nb, Ta, Al, Ti, Zr, and Hf), material compatibility with the insulating plug (e.g., the insulating plug 206), material compatibility with the potting material (e.g., the potting material 220), or material compatibility with the substrate (e.g., the substrate 230), as examples. Examples of suitable feedthrough ferrule materials may include titanium, niobium, aluminum, tantalum, zirconium, hafnium, or combinations thereof. Still further examples of suitable feedthrough ferrule materials may include metal alloys, such as an alloy of aluminum or alloys of suitable feedthrough ferrule materials described herein. It will be understood in light of the present disclosure that any suitable feedthrough ferrule material or combination of materials may be used, and the disclosure is not limited in this regard. It will be further understood in light of the present disclosure that suitable feedthrough ferrule materials may vary depending on factors, including those described herein.

In some embodiments as described herein, the implantable medical device feedthrough assembly may include one or more metal oxide surface coatings (e.g., the metal oxide surface coating 242). Each metal oxide surface coating (e.g., the first metal oxide surface coating 242 or the second metal oxide surface coating 244) may include or be any suitable metal oxide or combinations of metal oxides, which may be described as depending on the respective surface on which the metal oxide surface coating is disposed. For example, as described herein, a metal oxide surface coating may be disposed on a surface of a feedthrough pin. In embodiments including a metal oxide surface coating disposed on a surface of a feedthrough pin, the metal oxide surface coating may be formed by oxidizing at least a portion of the surface of the feedthrough pin. As another example, as described herein, a metal oxide surface coating may be disposed on a surface of a feedthrough ferrule. In embodiments including a metal oxide surface coating on a surface of a feedthrough ferrule, the metal oxide surface coating may be formed by oxidizing at least a portion of the surface of the feedthrough ferrule.

Suitable metal oxides may be selected based on biocompatibility, electrical insulation, material compatibility with the feedthrough pin (e.g., the feedthrough pin 202), material compatibility with the feedthrough ferrule (e.g., the feedthrough ferrule 204), or material compatibility with the potting material (e.g., the potting material 220), for example. Examples of suitable metal oxides may include titanium oxide, niobium oxide, aluminum oxide, tantalum oxide, zirconium oxide, hafnium oxide, or combinations thereof. It will be understood in light of the present disclosure that any suitable metal oxides or combinations of metal oxides may be used, and the disclosure is not limited in this regard. It will be further understood in light of the present disclosure that suitable metal oxides may vary depending on factors, including those described herein.

Each of the metal oxide surface coatings may have any suitable thickness. Thickness (to) of each metal oxide surface coating may be described, for example, as a dimension of the metal oxide surface coating (e.g., the metal oxide surface coating 242) along an axis orthogonal to a plane defined by an interface between the metal oxide surface coating and the surface on which it is disposed (e.g., the feedthrough ferrule 204). Thickness of a metal oxide coating may be measured, for example, using a calibrated color scheme based on the interferometric reflection of light from the metal surface through the transparent metal oxide. As another example, thickness (to) of each metal oxide surface coating may be described as a dimension of the metal oxide surface coating (e.g., the metal oxide surface coating 242) along an axis orthogonal to a plane tangential to an interface between the metal oxide surface coating and the surface on which it is disposed (e.g., a surface of the feedthrough pin 202). Suitable metal oxide thicknesses may be selected based on factors such as desired insulation, material compatibility with the potting material (e.g., the potting material 220), or structural stability of the metal oxide surface coating, as examples. Without wishing to be bound by theory, suitable metal oxide thicknesses may be thicker than a native oxide layer, which may be described as a thin layer of oxide (such as 10 nanometers (nm) or less) that may form on a metal surface at approximately ambient room temperature and ambient pressure of approximately 1 atmosphere (atm). In other words, and still without wishing to be bound by theory, a native oxide layer may be described as a thin layer of oxide formed through passive oxidation.

Suitable metal oxide thicknesses (to) may be or include between 10 nm and 300 nm or between 100 nm and 200 nm, for example, measured perpendicular to the longitudinal axis of feedthrough pin 202. In one embodiment, the metal oxide thickness may be approximately 150 nm. As further examples, suitable metal oxide thicknesses may be or include 10 nm or greater, 20 nm or greater, 30 nm or greater, 50 nm or greater, 70 nm or greater, 100 nm or greater, 120 nm or greater, 150 nm or greater, 180 nm or greater, 200 nm or greater, 250 nm or greater, or 300 nm or greater, and/or 300 nm or less, 250 nm or less, 200 nm or less, 180 nm or less, 150 nm or less, 120 nm or less, 100 nm or less, 70 nm or less, 50 nm or less, 30 nm or less, or 20 nm or less. It will be understood in light of the present disclosure that any suitable metal oxide thicknesses may be used, and the disclosure is not limited in this regard. It will be further understood in light of the present disclosure that suitable metal oxide thicknesses may vary depending on factors, including those described herein.

In one or more embodiments as described herein, the implantable medical device feedthrough assembly may include a potting material (e.g., the potting material 220 of the implantable medical device feedthrough assembly 200 shown in FIG. 2). The potting material may be or include any suitable materials or combination of materials. Suitable potting materials may be selected based on biocompatibility, electrical insulation, adhesion to and/or material compatibility with the feedthrough ferrule, adhesion to and/or material compatibility with the feedthrough pin, adhesion to and/or material compatibility with the substrate, adhesion to and/or material compatibility with each of the metal oxide surface coatings, as examples. As another example, suitable potting materials may be selected on desired curing temperature. Without wishing to be bound by theory, relatively lower potting material curing temperatures may be desirable to maintain the metal oxide surface coating while curing the potting material. Still without wishing to be bound by theory, metal oxide surface coatings may diffuse from a metal's surface (e.g., a surface of the feedthrough pin or a surface of the feedthrough ferrule) to the interior of the metal when exposed to sufficiently elevated temperatures. Suitable potting materials may include polymeric materials such as epoxies, adhesives, thermoplastics, or rubber, as examples. It will be understood in light of the present disclosure that any suitable potting materials or combination of potting materials may be used, and the disclosure is not limited in this regard. It will be further understood in light of the present disclosure that suitable potting materials may vary depending on factors, including those described herein.

In some embodiments as described herein, the implantable medical device feedthrough assembly may include an insulating plug (e.g., the insulating plug 206). The insulating plug may be or include any suitable materials or combination of materials. Suitable insulating plug materials may be selected based on factors such as insulation, biocompatibility, material compatibility with the potting material (e.g., the potting material 220), material compatibility with the substrate (e.g., the substrate 230), material compatibility with the feedthrough pin (e.g., the feedthrough pin 202), or material compatibility with the feedthrough ferrule (e.g., the feedthrough ferrule 204), as examples. Suitable insulating plug materials may include glass, sapphire, alumina, or zirconia for example. It will be understood in light of the present disclosure that any suitable insulating plug materials may be used, and the disclosure is not limited in this regard. It will be further understood in light of the present disclosure that suitable insulating plug materials may vary depending on factors, including those described herein.

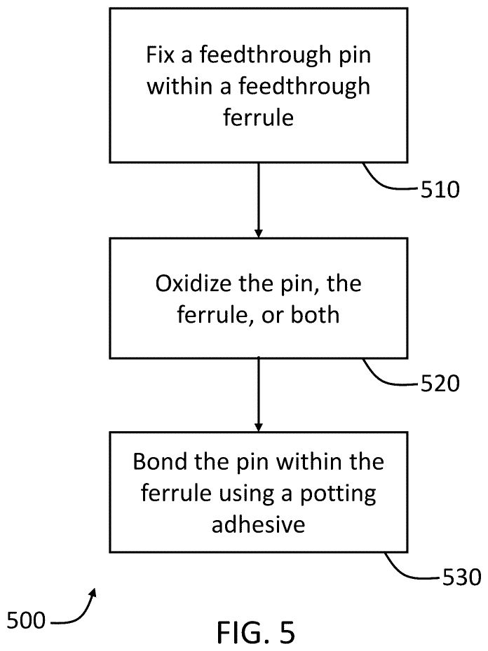

A method for forming a feedthrough assembly according to illustrative embodiments described herein may be provided. An illustrative method 500 of forming a feedthrough assembly is shown in FIG. 5. The method 500 may include fixing 510 a feedthrough pin (e.g., the feedthrough pin 202) within a feedthrough ferrule (e.g., the feedthrough ferrule 204) using an insulating plug (e.g., the insulating plug 206.

In one or more embodiments, the method 500 may include oxidizing 520 one or both of the feedthrough pin and the feedthrough ferrule. The oxidizing 520 may include oxidizing at least a portion of a surface of the feedthrough pin, at least a portion of a surface of the feedthrough ferrule, or at least a portion of a surface of each of the feedthrough pin and the feedthrough ferrule. In embodiments including oxidizing each of the feedthrough pin and the feedthrough ferrule, the oxidizing 520 may occur in any suitable sequence. For example, the oxidizing 520 may include oxidizing the feedthrough pin and subsequently oxidizing the feedthrough ferrule, or vice versa. Additionally or alternatively, the oxidizing 520 may include oxidizing the feedthrough pin and oxidizing the feedthrough ferrule concurrently or at least partially concurrently. The oxidizing 520 may form metal oxide surface layer (e.g., the metal oxide surface coating 242) disposed between the feedthrough pin and the feedthrough ferrule. The metal oxide surface coating may provide electrical insulation between the pin and the ferrule, such as electrical insulation secondary or supplementary to electrical insulation provided by the potting adhesive and the insulative plug, as described herein.

Any suitable oxidizing process may be used for the oxidizing 520. Suitable oxidizing processes may be selected based on factors such as desired thickness of each of the one or more metal oxide surface coatings, desired metal oxide or metal oxides of each of the one or more metal oxide surface coatings, as examples. Suitable oxidizing processes may include anodizing, as an example. Anodizing may be described as applying a voltage across a surface to be anodized (e.g., a surface of the feedthrough pin 202, a surface of the feedthrough ferrule 204, etc.) while the surface is in contact with an electrolyte (e.g., immersed in an electrolyte bath). For further examples, suitable oxidizing processes may include high temperature oxidation, atomic layer deposition (ALD), or sputtering, for example. In embodiments including oxidizing both the feedthrough pin and the feedthrough ferrule, the oxidizing 520 may include oxidizing each of the pin and the ferrule using the same oxidizing process. Additionally or alternatively, the oxidizing 520 may include oxidizing each of the feedthrough pin and the feedthrough ferrule using different oxidizing processes. It will be understood in light of the present disclosure that any suitable oxidizing process or combination of suitable oxidizing processes may be used, and the disclosure is not limited in this regard. It will be further understood in light of the present disclosure that suitable oxidizing processes may vary depending on factors, including those described herein.

In embodiments of the method 500 including high temperature oxidizing, or dry oxidizing, the oxidizing 520 may occur at any suitable ambient temperature. Suitable oxidizing ambient temperatures may be selected based on factors such as the feedthrough pin material composition, the feedthrough ferrule material composition, the desired metal oxide surface coating thickness, the desired material composition of the metal oxide surface coating, the duration of the high temperature oxidizing, as examples. Suitable oxidizing ambient temperatures may include between 20° C. and 500° C. or between 20° C. and 300° C., as examples. In one embodiment, the oxidizing 520 may occur at an ambient temperature of approximately 400° C. and at an ambient pressure of about 1 atmosphere (atm). It will be understood in light of the present disclosure that any suitable oxidizing ambient temperatures may be used, and the disclosure is not limited in this regard. It will be further understood in light of the present disclosure that suitable oxidizing ambient temperatures may vary depending on factors, including those described herein.

In embodiments of the method 500 including high temperature oxidizing, the oxidizing 520 may occur for any suitable duration, which may be defined as a time period during which the surface to be oxidized is in a target oxidizing ambient temperature environment, for example. Suitable high temperature oxidizing durations may be selected based on factors such as the oxidizing ambient temperature, the desired metal oxide surface coating thickness, or the desired material composition of the metal oxide surface coating, as examples. Suitable high temperature oxidizing durations may include between 5 minutes and 5 hours or between 30 minutes and 3 hours, as examples. In one embodiment, the oxidizing 520 may occur for a duration of approximately 1 hour. It will be understood in light of the present disclosure that any suitable high temperature oxidizing durations may be used, and the disclosure is not limited in this regard. It will be further understood in light of the present disclosure that suitable high temperature oxidizing durations may vary depending on factors, including those described herein.

In embodiments of the method 500 including anodizing, the oxidizing 520 may include contacting the surface to be anodized (e.g., a surface of the feedthrough pin 202, a surface of the feedthrough ferrule 204, or both) with an electrolyte. Any suitable electrolyte may be used. Suitable electrolytes may be selected based on factors such as material compatibility with the feedthrough ferrule, material compatibility with the feedthrough pin, material compatibility with the substrate, material compatibility with the insulating plug, the desired material composition of the metal oxide surface coating, the desired porosity of the metal oxide surface coating, or the desired thickness of the metal oxide surface coating, as examples. Suitable electrolytes may be or include, for example, sulfuric acid, phosphoric acid, borate, acetic acid, or combinations thereof. It will be understood in light of the present disclosure that any suitable electrolytes may be used, and the disclosure is not limited in this regard. It will be further understood in light of the present disclosure that suitable electrolytes may vary depending on factors, including those described herein.

In embodiments including anodizing, or wet oxidizing, the oxidizing 520 may include applying a voltage across the surface to be anodized. Any suitable anodizing voltage may be used, such as a positive DC voltage or an AC voltage (for example, with suitable positive and negative amplitudes), for example. Suitable anodizing voltages may include be selected based on factors such as the desired thickness of the metal oxide surface coating or the anodization constant of the surface to be oxidized, as just two examples. Without wishing to be bound by theory, a higher anodizing voltage (that is, a higher voltage amplitude) may generally afford a thicker metal oxide surface coating. For another example, suitable anodizing voltages may be selected based on the material composition of the surface to be anodized. Suitable DC anodizing voltages may include between 10 V and 200 V or between 20 V and 300 V, as examples. In one embodiment, the anodizing voltage may be approximately 150 V. It will be understood in light of the present disclosure that any suitable anodizing voltages may be used, and the disclosure is not limited in this regard. It will be further understood in light of the present disclosure that suitable anodizing voltages may vary depending on factors, including those described herein.

In at least one embodiment, the method 500 may include bonding 530 the feedthrough pin within the feedthrough ferrule with a potting material (e.g., the potting material 220). The potting material may provide electrical insulation between the feedthrough pin and the feedthrough ferrule, as described herein.

### Illustrative Aspects

The following is a list of illustrative embodiments according to the present disclosure.

Aspect 1 is an implantable medical device feedthrough assembly comprising:
a substrate having (i) a first major surface, and (ii) an opposing second major surface, wherein the first major surface is configured to be positioned towards an internal volume of the implantable medical device, and wherein the second major surface is configured to be positioned away from the internal volume of the implantable medical device;
a feedthrough ferrule having a ferrule sidewall defining a ferrule lumen extending through the substrate from the first major surface to the second major surface;
a feedthrough pin disposed within the ferrule lumen and configured to electrically connect a component in the internal volume of the implantable medical device with a component in communication with an environment external to the implantable medical device;
at least one metal oxide surface coating on a portion of at least one of the ferrule sidewall or a surface of the feedthrough pin to provide electrical insulation between the feedthrough pin and the feedthrough ferrule;
a potting material disposed between and providing electrical insulation between the feedthrough pin and the feedthrough ferrule, the potting material positioned to separate the internal volume from the external environment; and
an insulating plug disposed at least partially within the feedthrough ferrule and positioned to separate the potting material from the internal volume.

Aspect 2 is the feedthrough assembly of aspect 1, wherein the metal oxide surface coating pin from between the ferrule sidewall and the surface of the feedthrough pin, and extends along at least one of the ferrule sidewall or the surface of the feedthrough pin from the second major surface of the substrate towards the first major surface of the substrate.

Aspect 3 is the feedthrough assembly of aspect 2, wherein the metal oxide surface coating extends along the ferrule sidewall in the ferrule lumen from a major surface of the insulating plug towards the second major surface of the substrate.

Aspect 4 is the feedthrough assembly of any one of aspects 1 to 3, wherein the metal oxide surface coating is disposed on the ferrule sidewall.

Aspect 5 is the feedthrough assembly of any one of aspects 1 to 4, wherein the metal oxide surface coating is disposed on the surface of the feedthrough pin.

Aspect 6 is the feedthrough assembly of any one of aspects 1 to 5, wherein at least one metal oxide surface coating comprises a first metal oxide surface coating disposed on the surface of the feedthrough pin, and a second metal oxide surface coating disposed on the ferrule sidewall.

Aspect 7 is the feedthrough assembly of any one of aspects 1 to 6, wherein the feedthrough pin comprises titanium, niobium, aluminum, tantalum, zirconium, hafnium, or a combination thereof.

Aspect 8 is the feedthrough assembly of any one of aspects 1 to 7, wherein the feedthrough ferrule comprises titanium, niobium, aluminum, tantalum, or a combination thereof.

Aspect 9 is the feedthrough assembly of any one of aspects 1 to 8, wherein the metal oxide surface coating comprises titanium oxide, niobium oxide, aluminum oxide, tantalum oxide, zirconium oxide, hafnium oxide or a combination thereof.

Aspect 10 is the feedthrough assembly of any one of aspects 1 to 9, wherein the metal oxide surface coating has a thickness of 10 nm or greater, 20 nm or greater, 30 nm or greater, 50 nm or greater, or 100 nm or greater measured in a direction perpendicular to a longitudinal axis of the feedthrough pin.

Aspect 11 is the feedthrough assembly of any one of aspects 1 to 10, wherein the metal oxide surface coating has a thickness of between 50 nm and 300 nm or between 100 nm and 200 nm measured in a direction perpendicular to a longitudinal axis of the feedthrough pin.

Aspect 12 is the feedthrough assembly of any one of aspects 1 to 11, wherein the insulating plug comprises glass, sapphire, alumina, or a combination thereof.

Aspect 13 is an implantable medical device comprising:
the feedthrough assembly of any one of aspects 1 to 12;
a housing defining the internal volume of the implantable medical device, wherein the feedthrough assembly is fixed relative to the housing;
an internal component electrically coupled to the feedthrough pin and positioned in the internal volume; and
a second component electrically coupled to the feedthrough pin and in communication with the external environment.

Aspect 14 is the implantable medical device of aspect 13, further comprising:
a connector header configured to receive a medical lead,
wherein the second component is an electrical contact within the connector header, wherein the electrical contact is positioned and configured to electrically couple the lead to the internal component via the feedthrough pin.

Aspect 15 is a method for forming an implantable medical device feedthrough assembly, the method comprising:
fixing a feedthrough pin within a feedthrough ferrule with an insulating plug;
oxidizing one or both of the feedthrough pin and the feedthrough ferrule, thereby forming at least one metal oxide surface coating on a portion of at least one of the ferrule sidewall or a surface of the feedthrough pin to provide electrical insulation between the feedthrough pin and the feedthrough ferrule; and
bonding the pin within the ferrule with a potting material, the potting material providing electrical insulation between the feedthrough pin and the feedthrough ferrule.

Aspect 16 is the method of aspect 15, wherein the oxidizing occurs at an ambient temperature of between 200° C. and 500° C. or between 150° C. and 450° C.

Aspect 17 is the method of any one of aspects 15-16, wherein the oxidizing comprises applying a voltage across the feedthrough pin.

Aspect 18 is the method of aspect 17, wherein the voltage is between 50 V and 400 V or between 100 V and 450 V.

Aspect 19 is the method of any one of aspects 15 to 17, wherein the oxidizing comprises applying a voltage across the feedthrough ferrule.

Aspect 20 is the method of aspect 19 wherein the voltage is between 50 V and 400 V or between 100 V and 450 V.

Aspect 21 is the method of any one of aspects 15 to 20, wherein the metal oxide surface coating comprises titanium oxide, niobium oxide, aluminum oxide, tantalum oxide, or a combination thereof.

Aspect 22 is the method of any one of aspect 15 to 21, wherein the metal oxide surface coating has a thickness of 10 nm or greater, 20 nm or greater, 30 nm or greater, 50 nm or greater, or 100 nm or greater measured in a direction perpendicular to a longitudinal axis of the feedthrough pin.

Aspect 23 is the method of any one of aspects 15 to 22, wherein the metal oxide surface coating has a thickness of between 50 nm and 300 nm or between 100 nm and 200 nm measured in a direction perpendicular to a longitudinal axis of the feedthrough pin.

Aspect 24 is the method of any one of aspects 15 to 23, wherein the metal oxide surface coating is formed on the surface of the feedthrough pin.

Aspect 25 is the method of any one of aspects 15 to 24, wherein the metal oxide surface coating is formed on the ferrule sidewall.

Aspect 26 is the method of any one of aspects 15 to 25, wherein the feedthrough pin comprises titanium, niobium, aluminum, tantalum, or a combination thereof.

Aspect 27 is the method of any one of aspects 15 to 26, wherein the feedthrough ferrule comprises titanium, niobium, aluminum, tantalum, or a combination thereof.

All publications and patent applications in this specification are indicative of the level of ordinary skill in the art to which this technology pertains. All publications and patent applications are herein incorporated by reference to the same extent as if each individual publication or patent application was specifically and individually indicated by reference. In the event that any inconsistency exists between the disclosure of the present application and the disclosure(s) of any document incorporated herein by reference, the disclosure of the present application shall govern.

This application is intended to cover adaptations or variations of the present subject matter. It is to be understood that the above description is intended to be illustrative, and not restrictive, and the claims are not limited to the illustrative embodiments as set forth herein.

What is claims is:

1. An implantable medical device feedthrough assembly comprising:
a substrate having (i) a first major surface, and (ii) an opposing second major surface, wherein the first major surface is configured to be positioned towards an internal volume of the implantable medical device, and wherein the second major surface is configured to be positioned away from the internal volume of the implantable medical device;
a feedthrough ferrule having a ferrule sidewall defining a ferrule lumen extending through the substrate from the first major surface to the second major surface;
a feedthrough pin disposed within the ferrule lumen and configured to electrically connect a component in the internal volume of the implantable medical device with a component in communication with an environment external to the implantable medical device;
at least one metal oxide surface coating on a portion of at least one of the ferrule sidewall or a surface of the feedthrough pin to provide electrical insulation between the feedthrough pin and the feedthrough ferrule;
a potting material disposed between and providing electrical insulation between the feedthrough pin and the feedthrough ferrule, the potting material positioned to separate the internal volume from the external environment; and
an insulating plug disposed at least partially within the feedthrough ferrule and positioned to separate the potting material from the internal volume.

2. The feedthrough assembly of claim 1, wherein the metal oxide surface coating is disposed between the ferrule sidewall and the surface of the feedthrough pin and extends along at least one of the ferrule sidewall or the surface of the feedthrough pin from the second major surface of the substrate towards the first major surface of the substrate.

3. The feedthrough assembly of claim 2, wherein the metal oxide surface coating extends along the ferrule sidewall in the ferrule lumen from a major surface of the insulating plug towards the second major surface of the substrate.

4. The feedthrough assembly of claim 1, wherein the metal oxide surface coating is disposed on the ferrule sidewall.

5. The feedthrough assembly of claim 1, wherein the metal oxide surface coating is disposed on the surface of the feedthrough pin.

6. The feedthrough assembly of claim 1, wherein at least one metal oxide surface coating comprises a first metal oxide surface coating disposed on the surface of the feedthrough pin, and a second metal oxide surface coating disposed on the ferrule sidewall.

7. The feedthrough assembly of claim 1, wherein the feedthrough pin comprises titanium, niobium, aluminum, tantalum, zirconium, hafnium, or a combination thereof.

8. The feedthrough assembly of claim 1, wherein the feedthrough ferrule comprises titanium, niobium, aluminum, tantalum, or a combination thereof.

9. The feedthrough assembly of claim 1, wherein the metal oxide surface coating comprises titanium oxide, niobium oxide, aluminum oxide, tantalum oxide, zirconium oxide, hafnium oxide or a combination thereof.

10. The feedthrough assembly of claim 1, wherein the metal oxide surface coating has a thickness of 10 nm or greater measured in a direction perpendicular to a longitudinal axis of the feedthrough pin.

11. The feedthrough assembly of claim 1, wherein the metal oxide surface coating has a thickness of between 50 nm and 300 nm measured in a direction perpendicular to a longitudinal axis of the feedthrough pin.

12. The feedthrough assembly of claim 1, wherein the insulating plug comprises glass, sapphire, alumina, or a combination thereof.

13. An implantable medical device comprising:
the feedthrough assembly of claim 1;
a housing defining the internal volume of the implantable medical device, wherein the feedthrough assembly is fixed relative to the housing;
an internal component electrically coupled to the feedthrough pin and positioned in the internal volume; and
a second component electrically coupled to the feedthrough pin and in communication with the external environment.

14. The implantable medical device of claim 13, further comprising:
a connector header configured to receive a medical lead,
wherein the second component is an electrical contact within the connector header, wherein the electrical contact is positioned and configured to electrically couple the lead to the internal component via the feedthrough pin.

15. A method for forming an implantable medical device feedthrough assembly, the method comprising:
fixing a feedthrough pin within a feedthrough ferrule with an insulating plug;
oxidizing one or both of the feedthrough pin and the feedthrough ferrule, thereby forming at least one metal oxide surface coating on a portion of at least one of the ferrule sidewall or a surface of the feedthrough pin to provide electrical insulation between the feedthrough pin and the feedthrough ferrule; and
bonding the pin within the ferrule with a potting material, the potting material providing electrical insulation between the feedthrough pin and the feedthrough ferrule.

16. The method of claim 15, wherein the oxidizing occurs at an ambient temperature of between 200° C. and 500° C. or between 150° C. and 450° C.

17. The method of claim 15, wherein the oxidizing comprises applying a voltage across the feedthrough pin or across the feedthrough ferrule between 50 V and 400 V.

18. The method of claim 15, wherein the metal oxide surface coating has a thickness of between 50 nm and 300 nm measured in a direction perpendicular to a longitudinal axis of the feedthrough pin.

19. The method of claim 15, wherein the metal oxide surface coating is formed on the surface of the feedthrough pin.

20. The method of claim 15, wherein the metal oxide surface coating is formed on the ferrule sidewall.
